(19) United States
(12) Patent Application Publication
(10) Pub. No.: US 2017/0202838 A1
(43) Pub. Date: Jul. 20, 2017

(54) IMMUNOMODULATOR

(71) Applicant: TAIHO PHARMACEUTICAL CO., LTD., Tokyo (JP)

(72) Inventors: Kazutaka MIYADERA, Tokyo (JP); Satoshi FUKAYA, lbaraki (JP); Yoshimi AOYAGI, lbaraki (JP)

(73) Assignee: TAIHO PHARMACEUTICAL CO., LTD., Tokyo (JP)

(21) Appl. No.: 15/337,682

(22) Filed: Oct. 28, 2016

(30) Foreign Application Priority Data
Oct. 14, 2015 (JP) 2015-203282
Jul. 28, 2016 (JP) 2016-148854

(51) Int. Cl. A61K 31/519; A61K 39/39

(52) U.S. Cl. CPC A61K 31/519; A61K 39/39; A61K 2039/55511

(57) ABSTRACT

This invention relates to an immunomodulator comprising, as an active ingredient, (S)—N-(4-amino-5-(quinolin-3-yl)-6,7,8,9-tetrahydropyrimido[5,4-b]indolizin-8-yl)acrylamide represented by Formula (I) or a salt thereof and a pharmaceutical composition for the prevention or treatment of a disease that can be ameliorated via immunomodulation.

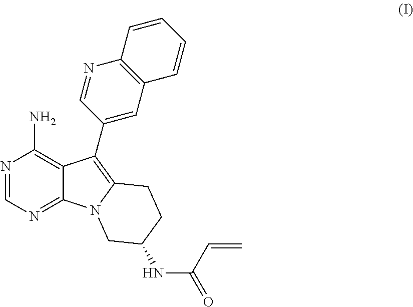 [SMILES: C=CC(=O)N[C@H]1CCc2c(-c3cnc4ccccc4c3)c3c(N)ncnc3n2C1]

### CROSS REFERENCE TO RELATED APPLICATIONS

This application claims priority to JP Patent Application No. 2016-148854, filed on Jul. 28, 2016, which claims priority to JP Patent Application No. 2015-203282, filed on Oct. 14, 2015.

### TECHNICAL FIELD

The present invention relates to a pharmaceutical composition for the prevention or treatment of a disease that can be ameliorated via immunomodulation, comprising, as an active ingredient, (S)—N-(4-amino-5-(quinolin-3-yl)-6,7,8,9-tetrahydropyrimido[5,4-b]indolizin-8-yl)acrylamide (hereinafter, this compound is also referred to as “Compound (I)”) or a salt thereof.

### BACKGROUND ART

The immune system has an important self-defense mechanism against various diseases caused by various factors inside and outside an organism. An impaired immune system adversely affects diseases, such as infections with bacteria or viruses, tumor incidence, and delayed recovery from injury or illness. Accordingly, modulation of the immune system is very critical for the prevention or treatment of various diseases. In the past, vaccination by means of administration of killed bacteria or antigens had been known as an immunomodulation method, and examples of other known techniques include methods involving the use of peptidoglycan, lipopolysaccharide, chitin, lactoferrin, or cyclophosphamide. In recent years, for example, cytokine therapy aimed at modulation of the immune system via administration of a protein such as IL-6, TNF, or IFN and immune cell therapy comprising sampling immune cells, exciting the activity thereof, and putting the cells back into the organism, have been possible. Such treatment techniques have exerted effects on the prevention or treatment of particular types of infections or tumors.

Epidermal growth factor receptor (EGFR) plays a very critical role in tumor growth. At present, various EGFR tyrosine kinase inhibitors have been developed and put into use in clinical settings. Specific examples of known EGFR tyrosine kinase inhibitors include: gefitinib (trade name: Iressa); erlotinib (trade name: Tarceva); and afatinib (trade name: Gilotrif). Such inhibitors are considered to have highly selective inhibitory activity against EGFR tyrosine kinase. In particular, such inhibitors are considered to exert anti-tumor effects on a subject having a mutation in the EGFR gene in a tumor-selective manner and contribute to improvement of the prognosis of the subject. In recent years, in addition, third-generation EGFR tyrosine kinase inhibitors exerting effects on T790M mutation, which is a resistance mechanism, and exhibiting enhanced selectivity to mutant EGFR (a representative example being AZ9291) have been developed. It is not too exaggeration to say that the anti-tumor effects of such compounds are achieved solely by highly selective inhibitory activity against EGFR tyrosine kinase, since such activity is capable of directly affecting tumor cells (Nature Rev. Cancer, vol. 6, pp. 803-811, 2006; and Journal of Thoracic Oncology, Vol. 3, No. 6, Supplement 2, June 2008).

Compound (I) and a salt thereof are known to inhibit EGFR tyrosine kinase with high selectivity and suppress tumor growth (WO 2013/125709). However, neither the compound of the present invention nor any of the known EGFR tyrosine kinase inhibitors described above are known to have immunomodulation activities.

### SUMMARY OF THE INVENTION

The object of the present invention is to provide an immunomodulator and a pharmaceutical composition for the prevention or treatment of a disease that can be ameliorated via immunomodulation.

The object of the present invention is also to provide a method for the prevention or treatment of a disease that can be ameliorated via immunomodulation with the immunomodulator and the pharmaceutical composition.

The present inventors have conducted concentrated studies concerning pharmacological activities of Compound (I). As a result, they discovered that such compound had activities of modulating the immune system, thereby completing the present invention. The present invention is summarized as follows.

(1) An immunomodulator comprising (S)—N-(4-amino-5-(quinolin-3-yl)-6,7,8,9-tetrahydropyrimido[5,4-b]indolizin-8-yl)acrylamide or a salt thereof.
(2) The immunomodulator according to (1), which activates T cells.
(3) The immunomodulator according to (1), which induces IL-2 production.
(4) The immunomodulator according to (1), which induces IFN production.
(5) The immunomodulator according to (1), which induces immunocyte migration.
(6) The immunomodulator according to (1), which induces exudation and accumulation of immunocytes in an affected area.
(7) A method for modulating the immune system of a subject, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
(8) A pharmaceutical composition for the prevention or treatment of an infection via immunomodulation, which comprises (S)—N-(4-amino-5-(quinolin-3-yl)-6,7,8,9-tetrahydropyrimido[5,4-b]indolizin-8-yl)acrylamide or a salt thereof.
(9) The pharmaceutical composition according to (8), wherein the infection is an infection with a parasite.
(10) The pharmaceutical composition according to (9), wherein the parasite is selected from the group consisting of trypanosomatid protozoa, malarial parasites, and Toxoplasma.
(11) The pharmaceutical composition according to (8), wherein the infection is an infection with a bacterium.
(12) The pharmaceutical composition according to (11), wherein the bacterium is selected from the group consisting of Streptococcus pneumoniae, Mycobacterium tuberculosis, Staphylococcus aureus, Bacillus anthracis, Vibrio cholerae, and Helicobacter pylori.
(13) The pharmaceutical composition according to (8), wherein the infection is an infection with a virus.
(14) The pharmaceutical composition according to (13), wherein the virus is selected from the group consisting of human T cell leukemia virus, papilloma virus, Epstein-Barr virus, cytomegalovirus, influenza virus, hepatitis B virus, and hepatitis C virus.
(15) A method for the prevention or treatment of an infection of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
(16) A pharmaceutical composition for the treatment of an immunodeficiency via immunomodulation, which comprises (S)—N-(4-amino-5-(quinolin-3-yl)-6,7,8,9-tetrahydropyrimido[5,4-b]indolizin-8-yl)acrylamide or a salt thereof.
(17) The pharmaceutical composition according to (16), wherein the immunodeficiency is caused by an infection with HIV.
(18) A method for the treatment of an immunodeficiency of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
(19) A pharmaceutical composition for the prevention or treatment of a disease caused by an immune system weakened with age via immunomodulation, comprising (S)—N-(4-amino-5-(quinolin-3-yl)-6,7,8,9-tetrahydropyrimido[5,4-b]indolizin-8-yl)acrylamide or a salt thereof.
(20) The pharmaceutical composition according to (19), wherein the disease caused by the weakened immune system is pneumonia.
(21) A method for the prevention or treatment of a disease caused by an immune system weakened with age of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
(22) A pharmaceutical composition for the prevention or treatment of a virus-associated tumor via immunomodulation, comprising (S)—N-(4-amino-5-(quinolin-3-yl)-6,7,8,9-tetrahydropyrimido[5,4-b]indolizin-8-yl)acrylamide or a salt thereof.
(23) The pharmaceutical composition according to (22), wherein the virus-associated tumor is Burkitt's lymphoma, hepatic carcinoma, uterine cervix cancer, adult T cell leukemia, Kaposi's sarcoma, or head and neck cancer.
(24) A method for the prevention or treatment of a virus-associated tumor of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
(25) A pharmaceutical composition for the potentiation of the activity of a medicine used for preventing or treating a disease by acting on an immune system, comprising (S)—N-(4-amino-5-(quinolin-3-yl)-6,7,8,9-tetrahydropyrimido[5,4-b]indolizin-8-yl)acrylamide or a salt thereof.
(26) The pharmaceutical composition according to (25), which is used for the potentiation of the activity of a vaccine to prevent an infection.
(27) The pharmaceutical composition according to (25), which is used for the potentiation of the activity of an antiviral agent.
(28) The pharmaceutical composition according to (25), which is used for the potentiation of the activity of an anti-PD-1 antibody or an anti-PD-L1 antibody.
(29) The pharmaceutical composition according to (25), which is used for the potentiation of the activity of a cancer vaccine.
(30) The pharmaceutical composition according to (25), which is used for the potentiation of the activity of an agent for inducing an antitumor immune response.
(31) The pharmaceutical composition according to (30), wherein the agent for inducing an antitumor immune response is an anti-PD-1 antibody or an anti-PD-L1 antibody.
(32) The pharmaceutical composition according to (31), wherein the agent for inducing an antitumor immune response is an anti-PD-1 antibody.
(33) The pharmaceutical composition according to (31), wherein the agent for inducing an antitumor immune response is an anti-PD-L1 antibody.
(34) A method for the potentiation of the activity of a medicine used for preventing or treating a disease of a subject by acting on an immune system, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

The present invention provides an immunomodulator comprising, as an active ingredient, (S)—N-(4-amino-5-(quinolin-3-yl)-6,7,8,9-tetrahydropyrimido[5,4-b]indolizin-8-yl)acrylamide or a salt thereof and a novel pharmaceutical composition for the prevention or treatment of a disease that can be ameliorated via immunomodulation. The present invention also provides a novel method for the treatment of, for example, various infections, immunodeficiency, and tumors.

This description includes the disclosures of Japanese Patent Application Nos. 2015-203282 and 2016-148854, which are priority documents of the present application.

### BRIEF DESCRIPTION OF THE DRAWINGS

FIG. 1 shows the concentration of IL-2 in a culture supernatant produced by mouse spleen cells when the anti-CD3 antibody and the anti-CD28 antibody are added at constant levels and Compound (I) is added at various concentrations in Example 1.

FIG. 2 shows the concentration of IL-2 in a culture supernatant produced by mouse spleen cells with or without the addition of Compound (I), when the anti-CD3 antibody is added at various concentrations and the anti-CD28 antibody is added at a constant level in Example 1.

FIG. 3 shows changes in the concentration of IL-2 in a culture supernatant produced by human peripheral blood mononuclear cells, when Compound (I) is added or AZD9291 is added in Example 2.

FIG. 4 shows the extent of 3H-Thd uptake resulting when a mixed lymphocyte reaction is induced with the addition of Compound (I), AZD9291, or Erlotinib in Example 3.

FIG. 5 shows the results of flow cytometric analysis of the CFSE levels in CD4-positive and CD8-positive cells, when various compounds are added in Example 4.

FIG. 6 shows the results of calculation of relative numbers of CD4-positive cells, CD8-positive cells, and CD4- and CD8-negative and NK1.1-positive cells, when the anti-PD-1 antibody or Compound (I) is added in Example 5.

FIG. 7 shows the results of calculation of relative numbers of CD4-positive and CD69-positive cells and CD4-positive, CD44-positive, and CD62L-negative cells, when the anti-PD-1 antibody and/or Compound (I) is(are) added in Example 6.

FIG. 8 shows the results of calculation of the CD44 expression level in CD4-positive cells and the CD62L expression level in CD4-positive cells, when the anti-PD-1 antibody and/or Compound (I) is(are) added in Example 6.

FIG. 9 shows the concentration of cytokine produced by human peripheral blood mononuclear cells of the groups to which compounds are added in Example 7 relative to that of the control group.

FIG. 10 shows the number of pulmonary metastatic nodules of the groups to which Compound (I) had been administered in Example 8 observed 14 days after mouse melanoma implantation (i.e., 15 days after drug administration).

FIG. 11 shows changes in tumor volumes of MC38 tumor strains of the groups to which the anti-PD-1 antibody or Compound (I) had been administered with the elapse of time in Example 9.

FIG. 12 shows changes in tumor volumes of K1735M2 tumor strains of individuals to which the anti-PD-1 antibody, the anti-PD-L1 antibody, or Compound (I) had been administered alone or either the anti-PD-1 antibody or the anti-PD-L1 antibody had been administered in combination with Compound (I) with the elapse of time in Example 10.

FIG. 13 shows changes in tumor volumes of MC38 tumor strains of the groups to which the anti-PD-1 antibody or Compound (I) or the combination thereof had been administered with the elapse of time in Example 11.

FIG. 14 shows relative proportions of CD3, CD4, and CD8 gene expression levels in tumors sampled in Example 11.

FIG. 15 shows relative proportions of NK1.1, IL-2, and IFN-γ gene expression levels in tumors sampled in Example 11.

FIG. 16 shows relative proportions of Perforin, Granzyme B, and CD69 gene expression levels in tumors sampled in Example 11.

### EMBODIMENTS FOR CARRYING OUT THE INVENTION

Compound (I), i.e., (S)—N-(4-amino-5-(quinolin-3-yl)-6,7,8,9-tetrahydropyrimido[5,4-b]indolizin-8-yl)acrylamide, is represented by Structural Formula (I) below. Compound (I) is a known compound, and a method for producing the same is disclosed in WO 2013/125709.

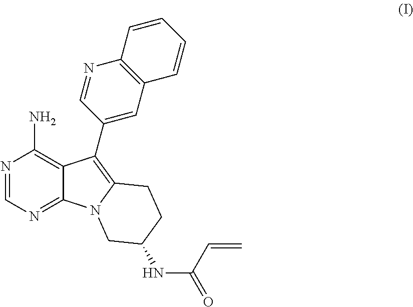 [SMILES: C=CC(=O)N[C@H]1CCc2c(-c3cnc4ccccc4c3)c3c(N)ncnc3n2C1]

Compound (I) may be in a free form or a salt form. When it is in a salt form, it may be in the form of a crystal. In such a case, a crystalline form may be a single crystal or a polymorphic mixture. Further, it may be a solvate (e.g., a hydrate) or a non-solvate. An example of a salt form is an acid addition salt, and specific examples thereof include: inorganic acid salts, such as salts of hydrochloric acid, sulfuric acid, nitric acid, phosphoric acid, and perchloric acid; sulfonic acid salts, such as salts of methanesulfonic acid, isethionic acid, benzenesulfonic acid, and p-toluenesulfonic acid; and other organic acid salts, such as salts of formic acid, maleic acid, fumaric acid, tartaric acid, citric acid, ascorbic acid, and trifluoroacetic acid.

Compound (I) and a salt thereof exert immunomodulation activity on subjects; that is, humans and other mammalians, such as monkeys, mice, rats, rabbits, dogs, cats, cows, horses, pigs, and sheep. Preferably, Compound (I) and a salt thereof exert immunomodulation activity on humans. The term “immunomodulation activity” used herein refers to activation of immunocytes. Specifically, the term refers to the activity of inducing division and differentiation of immunocytes, inducing the production of various cytokines, allowing immunocytes to migrate, and allowing the immunocytes to undergo exudation and/or accumulation in an affected area (i.e., a region in which a pathological change has occurred, such as a tumor tissue, infectious tissue, or inflammatory tissue); or enhancing the functions of immunocytes to eliminate foreign-matter-like components of endogenous origin or foreign matter of exogenous origin. Compound (I) and a salt thereof have activity of activating T cells, in particular, among immunocytes. Examples of induced cytokines include IL-1β, IL-2, IL-4, IL-5, IL-6, IL-8, IL-9, IL-17, IL-23, GM-CSF, IFN-γ, MCFA, MIP-1α, MIP-1β, and TNF-α, and a specific example is IL-2. In addition, Compound (I) and a salt thereof have activity of inducing cytokine production, particularly on peripheral blood mononuclear cells. Among various types of cytokines, in particular, production of IL-2 and/or IFN is induced. Also, Compound (I) and a salt thereof allow immunocytes to migrate. Further, Compound (I) and a salt thereof induce migration, exudation, and/or accumulation of immunocytes. Migration, exudation and/or accumulation of immunocytes in an affected area can be examined on the basis of tissue staining of the affected area and changes in expression levels of the gene characteristic to the immunocyte in the tissues of the affected area (examples of such genes include CD3, CD4, CD8, NK1.1, IL-2, IFN-γ, Perforin, Granzyme B, and CD69 genes). The present invention relates to: an immunomodulator containing Compound (I) or a salt thereof; Compound (I) or a salt thereof as an immunomodulator; and a method for immunomodulation of a subject comprising administering an effective amount of Compound (I) or a salt thereof to the subject in need of prevention or treatment.

The immunomodulation activity of Compound (I) and a salt thereof enables the prevention or treatment of various infections, immunodeficiency, diseases caused by an immune system weakened with age, and virus-associated tumors.

Specific examples of infections Compound (I) and a salt thereof can prevent or treat include infections with parasites (e.g., infections with parasites selected from the group consisting of trypanosomatid protozoa, malarial parasites, and Toxoplasma), infections with bacteria (e.g., infections with bacteria selected from the group consisting of Streptococcus pneumoniae, Mycobacterium tuberculosis, Staphylococcus aureus, Bacillus anthracis, Vibrio cholerae, Mycoplasmata, and Helicobacter pylori), and infections with viruses (e.g., infections with viruses selected from the group consisting of human T cell leukemia viruses (HTLV-1), papilloma viruses (HPV), Epstein-Barr viruses (EBV), cytomegaloviruses (CMV), influenza viruses (FLU), hepatitis B viruses (HBV), herpes virus, and hepatitis C viruses (HCV)). Other embodiments of the present invention relate to: a pharmaceutical composition for the prevention or treatment of an infection via immunomodulation, which contains Compound (I) or a salt thereof; Compound (I) or a salt thereof for the prevention or treatment of an infection via immunomodulation; and a method for the prevention or treatment of an infection of a subject via immunomodulation comprising administering an effective amount of Compound (I) or a salt thereof to the subject in need of prevention or treatment.

Specific examples of immunodeficiency Compound (I) and a salt thereof can treat include congenital immunodeficiency and acquired immunodeficiency, and a more specific example is acquired immunodeficiency caused by infection with human immunodeficiency virus (HIV). Accordingly, other embodiments of the present invention relate to: a pharmaceutical composition for the treatment of an immunodeficiency via immunomodulation, which contains Compound (I) or a salt thereof; Compound (I) or a salt thereof for the treatment of an immunodeficiency via immunomodulation; and a method for the treatment of an immunodeficiency of a subject via immunomodulation comprising administering an effective amount of Compound (I) or a salt thereof to the subject in need of treatment.

A specific example of a disease caused by an immune system weakened with age Compound (I) and a salt thereof can prevent or treat is pneumonia. Accordingly, other embodiments of the present invention relate to: a pharmaceutical composition for the prevention or treatment of a disease caused by an immune system weakened with age via immunomodulation, which contains Compound (I) or a salt thereof; Compound (I) or a salt thereof for the prevention or treatment of a disease caused by an immune system weakened with age via immunomodulation; and a method for the prevention or treatment of a disease caused by an immune system weakened with age of a subject via immunomodulation comprising administering an effective amount of Compound (I) or a salt thereof to the subject in need of prevention or treatment.

Specific examples of virus-associated tumors (i.e. tumors developed by infections with viruses) Compound (I) and a salt thereof can prevent or treat include Burkitt's lymphoma, hepatic carcinoma, uterine cervix cancer, adult T cell leukemia, Kaposi's sarcoma, and head and neck cancer. Accordingly, other embodiments of the present invention relate to: a pharmaceutical composition for the prevention or treatment of a virus-associated tumor via immunomodulation, which contains Compound (I) or a salt thereof; Compound (I) or a salt thereof for the prevention or treatment of a virus-associated tumor via immunomodulation; and a method for the prevention or treatment of a virus-associated tumor of a subject via immunomodulation comprising administering an effective amount of Compound (I) or a salt thereof to the subject in need of prevention or treatment.

The activity of Compound (I) and a salt thereof for immunomodulation can potentiate the effects of a medicine used for the prevention or treatment of a disease by acting on an immune system. Specific examples of medicines used for the prevention or treatment of a disease by acting on an immune system include: vaccines for the prevention of infections (e.g., vaccines for the prevention of infections such as diphtheria, tetanus, and pertussis), antiviral agents (e.g., influenza vaccines, hepatitis B vaccines, interferon α preparations, interferon β preparations, Telaprevir, Ribavirin, Simeprevir, Vidarabine, acyclovir, Ganciclovir, Valganciclovir, nucleoside analog reverse transcriptase inhibitors (NRTI) (e.g., AZT (zidovudine), ddI (Didanosine), ddC (Zalcitabine), d4T (Stavudine), and 3TC (lamivudine)), non-nucleoside reverse transcriptase inhibitors (NNRTI) (e.g., Nevirapine or Delavirdine), and protease inhibitors (e.g., Saquinavir, Ritonavir, Indinavir, or Nelfinavir)); agents for inducing antitumor immune responses (e.g., modulators having immunomodulation activity, such as CD28-like family members or CD28-like family ligand members: specific examples thereof include programmed death-1 (PD-1) inhibitors, programmed death-Ligand 1 (PD-L1) inhibitors, programmed death-Ligand 2 (PD-L2) inhibitors, anti-CTLA-4 inhibitors, anti-BTLA inhibitors, anti-CD28 modulators, anti-ICOS modulators, anti-ICOS-L modulators, anti-B7-1 modulators, anti-B7-2 modulators, anti-B7-H3 modulators, and anti-B7-H4 modulators; more specific examples thereof include anti-PD-1 antibodies, PD-1 peptide inhibitors, anti-PD-1 RNAi, anti-PD-1 antisense RNA, anti-PD-L1 antibodies, PD-L1 peptide inhibitors, anti-PD-L1 RNAi, anti-PD-L1 antisense RNA, anti-PD-L2 antibodies, PD-L2 peptide inhibitors, anti-PD-L2 RNAi, anti-PD-L2 antisense RNA, and anti-CTLA4 antibodies; and particularly specific examples thereof include anti-PD-1 antibodies and anti-PD-L1 antibodies), and cancer vaccines (e.g., Sipuleucel-T). Accordingly, other embodiments of the present invention relate to: a pharmaceutical composition for the potentiation of the activity of a medicine used for the prevention or treatment of a disease by acting on an immune system, which contains Compound (I) or a salt thereof; Compound (I) or a salt thereof for the potentiation of the activity of a medicine used for the prevention or treatment of a disease by acting on an immune system; and a method for the potentiation of the activity of a medicine used for the prevention or treatment of a disease by acting on an immune system comprising administering an effective amount of Compound (I) or a salt thereof in combination with such medicine to a subject.

The present invention also relates to use of Compound (I) or a salt thereof for the production of the immunomodulator and the pharmaceutical composition described above.

The immunomodulator and the pharmaceutical composition according to the present invention may comprise a pharmaceutically acceptable diluent, excipient, or adjuvant, according to need, and it may be prepared in a form adequate for an administration route. Specific examples of dosage forms include oral preparations (e.g., tablets, pills, capsules, granules, powders, and liquid preparations), injection preparations, suppositories, ointments, and adhesive skin patches. Such dosage forms can be prepared in accordance with conventional techniques. The immunomodulator and the pharmaceutical composition according to the present invention are preferably in the form of oral preparations because of the ease of administration. Specific examples of adjuvants include binders, disintegrators, lubricants, colorants, solubilizers, flavoring agents, suspending agents, isotonizing agents, buffers, and soothing agents. According to need, the immunomodulator and the pharmaceutical composition may contain additives, such as preservatives, antioxidants, colorants, sweeteners, and stabilizers.

The amount of the immunomodulator and the pharmaceutical composition according to the present invention to be administered varies depending on the purpose of administration, the age, gender, and body weight of the subject to which the immunomodulator and the pharmaceutical composition are to be administered, as well as the route of administration. In the case of an adult whose body weight is 50 kg, for example, Compound (I) or a salt thereof is administered in an amount of preferably 0.05 to 5,000 mg, and more preferably 0.1 to 1,000 mg per day. Administration can be performed, for example, once per two days, once per day, or two or three times per day.

Compound (I) and a salt thereof according to the present invention have the immunomodulation activity as described in the examples below, and Compound (I) and a salt thereof make contributions to the regulation of vital functions, health enhancement, and enhanced functions of eliminating foreign-matter-like components of endogenous origin or foreign matter of exogenous origin of humans and other mammalian animals.

### EXAMPLES

Hereafter, the present invention is described in greater detail with reference to the examples, although the technical scope of the present invention is not limited to these examples.

### [Example 1] Induction of Cytokine Production by Compound (I) of Mouse Spleen Cells Stimulated with Anti-CD3 Antibody and Anti-CD28 Antibody

The mouse spleen was extracted, grinded with glass slides with frosted areas, and then subjected to hemolysis, so as to obtain spleen cells. The resulting spleen cells were introduced into a complete medium (i.e., RPMI-1640 supplemented with 10% heat-inactivated FBS, 100 U/ml penicillin, 100 μg/ml streptomycin, and 55 μM 2-mercaptoethanol) to a cell density of 2×10⁶ cells/ml, the anti-CD3 antibody and the anti-CD28 antibody were added thereto to the final concentrations of 3 μg/ml and 0.5 μg/ml, respectively, and Compound (I) was further added thereto to each final concentration. The culture solution was seeded in a 96-well plate at 200 μl/well and then cultured in an incubator at 37° C. in the presence of 5% CO₂ for 2 days. The culture supernatant was recovered, and the concentration of IL-2 contained therein was measured via ELISA using anti-mIL-2 antibody.

Also, culture was conducted in the same manner with the addition of the anti-CD3 antibody at a variable final concentration, the anti-CD28 antibody at the final concentration of 0.5 μg/ml, and Compound (I) at the final concentration of 0.0 μM or 0.1 μM, and the IL-2 concentration in the culture supernatant was measured.

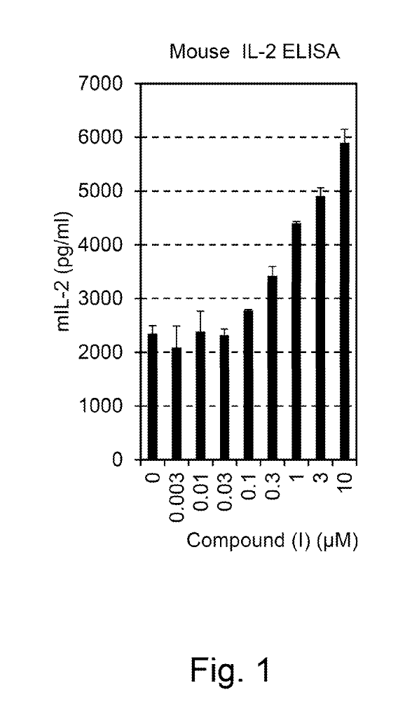

FIG. 1 shows a chart demonstrating changes in the concentration of IL-2 produced by mouse spleen cells in the culture supernatant, when the concentration of the anti-CD3 antibody and that of the anti-CD28 antibody were made constant and the concentration of Compound (I) was made variable. As demonstrated in the chart, the concentration of IL-2 produced by mouse spleen cells increased as the amount of Compound (I) added increased.

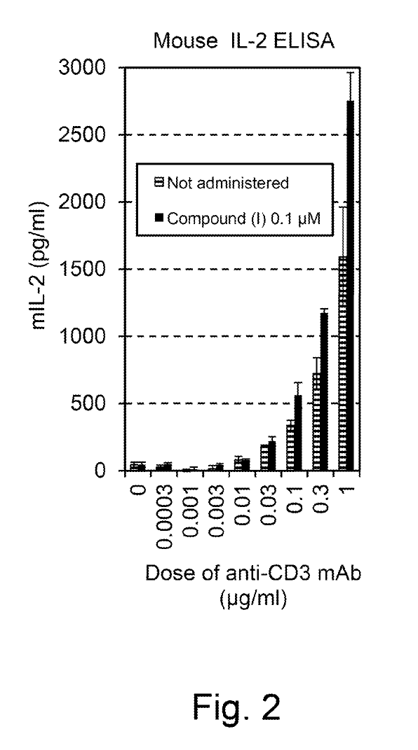

FIG. 2 shows a chart demonstrating differences in the concentration of IL-2 produced by mouse spleen cells in the culture supernatant caused by the addition of Compound (I), when the concentration of the anti-CD3 antibody was made variable and that of the anti-CD28 antibody was made constant. As demonstrated in the chart, IL-2 production was induced upon stimulation with the anti-CD3 antibody when Compound (I) was added, although an extent of induction was low when Compound (I) was not added.

These results demonstrate that Compound (I) could potentiate induction of IL-2 production of mouse spleen cells and that Compound (I) exerts immunomodulation activity.

### [Example 2] Induction of Cytokine Production by Compound (I) of Human Peripheral Blood Mononuclear Cells

Human peripheral blood mononuclear cells were introduced into a human complete medium (i.e., RPMI-1640 supplemented with 10% heat-inactivated FBS, 100 U/ml penicillin, and 100 μg/ml streptomycin) to prepare a cell suspension (1×10⁶ cells/ml). Phytohemagglutinin M (PHA-M) was added thereto to the final concentration of 5 μg/ml, and Compound (I) or an EGFR tyrosine kinase inhibitor (AZD9291) was further added thereto to each final concentration. This culture solution was seeded in a 96-well plate at 200 μl/well and then cultured in an incubator at 37° C. in the presence of 5% CO₂ for 3 days. The culture supernatant was recovered, and the concentration of IL-2 therein was measured via ELISA using the anti-hIL-2 antibody.

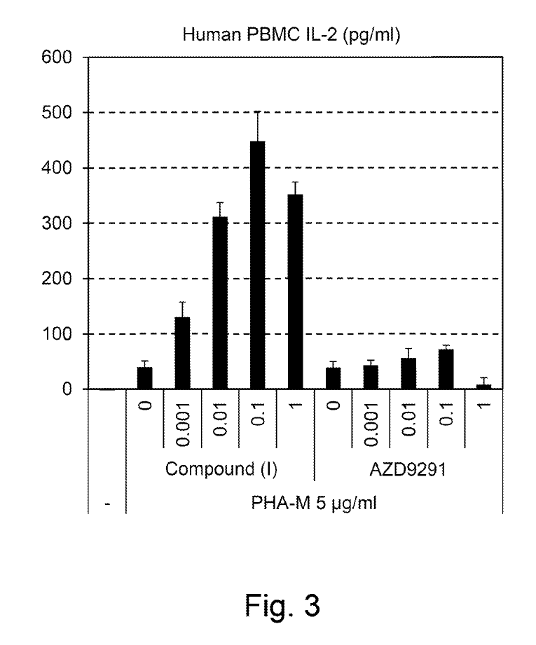

FIG. 3 shows a chart demonstrating changes in the concentration of IL-2 produced by human peripheral blood mononuclear cells in the culture supernatant, when Compound (I) was added and when AZD9291 was added, respectively. As shown in the chart, Compound (I) induced IL-2 production of the human peripheral blood mononuclear cells; however, AZD9291, which is an EGFR tyrosine kinase inhibitor as with Compound (I), exerted substantially no activity for inducing IL-2 production. These results demonstrate that Compound (I) has immunomodulation activity, which is inherent to Compound (I), and EGFR tyrosine kinase inhibitors does not share such activity.

### [Example 3] T Cell Modulation by Compound (I) Through Mixed Lymphocyte Reaction

A mixed lymphocyte reaction (MLR) is a representative experimentation technique for T cell modulation described in, for example, J. Exp. Med. 127 (5): 879-90, 1968. The influence of Compound (I) imposed on T cell modulation through this reaction was examined.

Spleens were extracted from C57BL/6N mice and BALB/c mice, grinded with glass slides with frosted areas, and then subjected to hemolysis, so as to obtain spleen cells. The C57BL/6N mouse spleen cells and the BALB/c mouse spleen cells were introduced into a complete medium (i.e., RPMI-1640 supplemented with 10% heat-inactivated FBS, 100 U/ml penicillin, 100 μg/ml streptomycin, and 55 μM 2-mercaptoethanol). The BALB/c mouse spleen cells were irradiated with 30 Gy X-ray to destroy the growth activity. The spleen cells of these allogeneic mice were each added to the final concentration of 1×10⁵ cells/well and mixed (Alo), and Compound (I) or the EGFR tyrosine kinase inhibitor (i.e., AZD09291 or Erlotinib) adjusted to relevant concentrations was added thereto. As a control sample, a mixture of syngeneic C57BL/6N mouse spleen cells (Syn) was also prepared. The culture solution was seeded in a 96-well plate at 200 μl/well and then cultured in an incubator at 37° C. in the presence of 5% CO₂ for 3 days. Tritium-labeled thymidine (3H-Thd) was added thereto 2 days after the initiation of culture. The amount of 3H-Thd uptake was measured using a liquid scintillation counter.

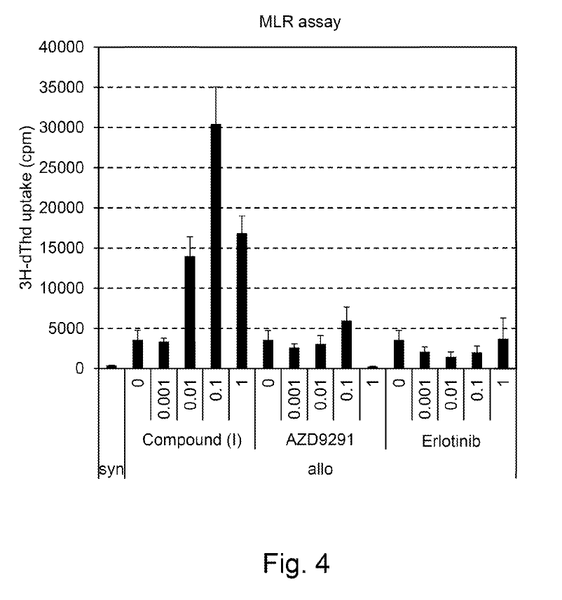

FIG. 4 shows a chart demonstrating the amount of 3H-Thd uptake for each compound at different concentrations, when the mixed lymphocyte reaction is induced. The amount of 3H-Thd uptake indicates the amount of T cells grown through the mixed lymphocyte reaction. As shown in the chart in FIG. 4, Compound (I) induced the growth of T cells derived from the C57BL/6N mice that would be excited through the mixed lymphocyte reaction. In contrast, such activity was not observed in other EGFR tyrosine kinase inhibitors. These results demonstrate that Compound (I) has immunomodulation activity, which is inherent to Compound (I), and the EGFR tyrosine kinase inhibitor does not have such activity.

### [Example 4] Excitation of T Cell Growth by Compound (I)

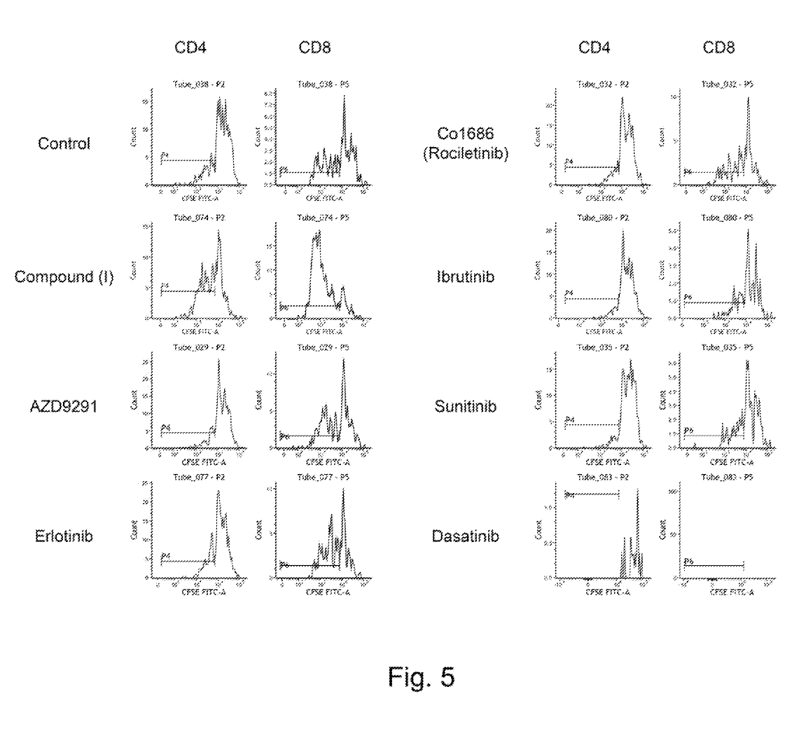

The mouse spleen was extracted, grinded with glass slides with frosted areas, and then subjected to hemolysis, so as to obtain spleen cells. The spleen cells were suspended in 5 ml of a staining buffer (0.5% BSA, 2 mM EDTA, PBS(-)), and the cells were stained with 5 μM CFSE (5-carboxyfluorescein succinimidyl ester). Thereafter, the stained cells were washed with an ice-cooled complete medium (RPMI-1640). The CFSE-stained spleen cells were introduced into a complete medium (i.e., RPMI-1640 supplemented with 10% heat-inactivated FBS, 100 U/ml penicillin, 100 μg/ml streptomycin, and 55 μM 2-mercaptoethanol) to prepare a cell suspension (1×10⁶ cells/ml), the anti-CD3 antibody and the anti-CD28 antibody were added at 1 μg/ml and 1 μg/ml, respectively, and Compound (I) adjusted to various concentrations or the EGFR tyrosine kinase inhibitor (i.e., AZD09291, Erlotinib, Co1686 (Rociletinib), Ibrutinib, Sunitinib, or Dasatinib) was added at 0.1 μM. A control sample that does not contain the EGFR tyrosine kinase inhibitor was also prepared. These culture solutions were seeded in a 96-well plate at 200 μl/well and then cultured in an incubator at 37° C. in the presence of 5% CO₂ for 3 days. The cells were recovered and stained with the anti-CD4 antibody and the anti-CD8 antibody. The amounts of CFSE in CD4- and CD8-positive cells were analyzed via flow cytometry. FIG. 5 shows the results of analysis.

Once CFSE is incorporated into a cell, the amount thereof is kept constant in the cell. Since the amount of CFSE per cell decreases by half upon cell division, according to flow cytometric analysis, the intensity of CFSE staining is attenuated and is shifted toward left (i.e., lower level) in the chart, as cell division proceeds. In the case of the sample to which Compound (I) had been added, as shown in FIG. 5, the amount of CFSE per CD4-positive cell and CD8-positive cell decreases. This indicates that the growth of such cells is enhanced. In the case of the samples to which other types of EGFR tyrosine kinase inhibitors had been added, in contrast, no such activity for enhancing the cell growth was observed. In the group to which Dasatinib had been added, cell death was induced. The results demonstrate that Compound (I) has immunomodulation activity, which is inherent to Compound (I), and other EGFR tyrosine kinase inhibitors do not have such activity.

### [Example 5] Analysis of Peripheral Blood Immunocyte in OVA-Expressing Mouse Model Subcutaneously Implanted with Thymoma Cell Line

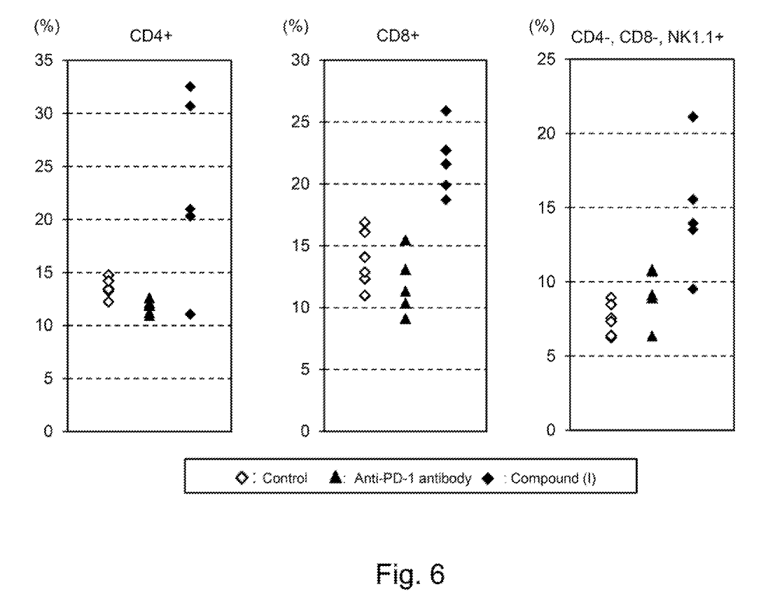

A cell suspension of the OVA-expressing mouse thymoma cell line (EG.7-OVA) was prepared with the use of PBS(-) and 50% Matrigel, and the cell suspension (1×10⁴ cells/mouse) was implanted into the syngeneic C57BL/6n mice via subcutaneous injection. The mice were divided into groups based on their body weights 1 day after implantation, and Compound (I) was administered thereto at 50 mg/kg or the anti-PD-1 antibody was administered thereto at 100 μg/mouse. A control group to which neither Compound (I) nor the anti-PD-1 antibody had been administered was also prepared. The peripheral blood was sampled 14 days after implantation and analyzed via flow cytometry with the use of antibodies each reacting with a relevant immunocyte surface marker, so as to determine the relative number of CD4-positive cells, CD8-positive cells, and CD4-negative, CD8-negative, and NK1.1-positive cells. FIG. 6 shows the results of analysis. In the group to which Compound (I) had been administered, the number of all cells increased, compared with the control group and the group to which the anti-PD-1 antibody had been administered. The results demonstrate that Compound (I) has activity of increasing the number of immunocyte subsets in vivo and has immunomodulation activity.

### [Example 6] Analysis of Immunocyte in Spleen Cells in Mouse Model Subcutaneously Implanted with Colon Cancer Cell Line

A cell suspension of the mouse colon cancer cell line (colon26) was prepared with the use of PBS(-) and 50% Matrigel, and the cell suspension was implanted into the syngeneic BALB/c mice via subcutaneous injection at 2×10³ cells/mouse. The mice were divided into groups based on their body weights 1 day after implantation, and Compound (I) was administered thereto at 50 mg/kg and/or the anti-PD-1 antibody was administered thereto at 100 μg/mouse. A control group to which neither Compound (I) nor the anti-PD-1 antibody had been administered was also prepared. The spleen cells were sampled 21 days after implantation and analyzed via flow cytometry with the use of antibodies each reacting with a relevant immunocyte surface marker.

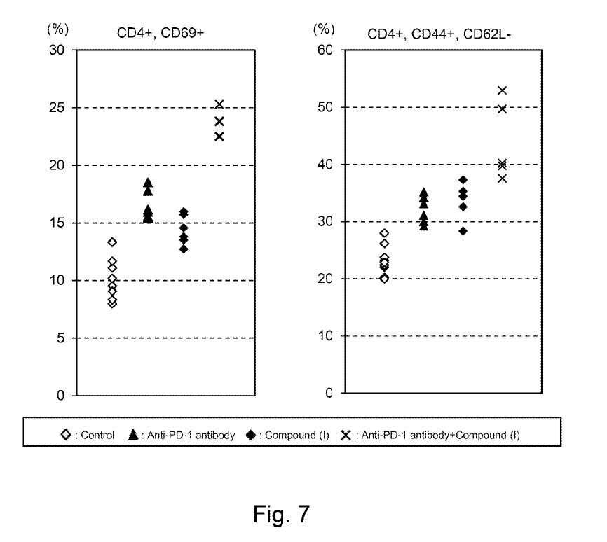

FIG. 7 shows a chart demonstrating the results of calculation of the relative numbers of CD4-positive and CD69-positive cells and CD4-positive, CD44-positive, and CD62L-negative cells. In the group to which Compound (I) had been administered, the number of the CD4-positive and CD69-positive cells and that of the CD4-positive, CD44-positive, and CD62L-negative cells were greater than those in the control group. In the group to which Compound (I) had been administered in combination with the anti-PD-1 antibody, the number of such cells was apparently greater.

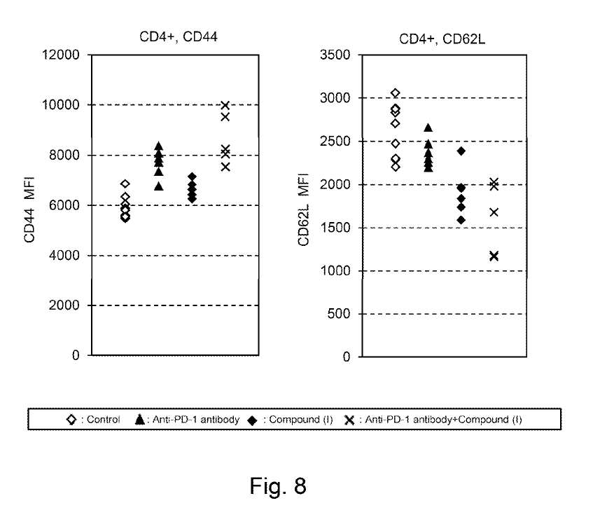

FIG. 8 shows a chart demonstrating the results of calculation of the CD44 expression level in CD4-positive cells and the CD62L expression levels in CD4-positive cells. The CD44 expression level and the CD62L expression level are each represented in terms of the mean fluorescence intensity (MFI) of the surface marker. Compared with the control group, the CD44 expression level increased and the CD62L expression level decreased in the CD4-positive cells of the group to which Compound (I) had been administered. In the group to which Compound (I) had been administered in combination with the anti-PD-1 antibody, in addition, such phenomena were more apparent. The results indicate that the number of effector memory T cells in the spleen cells that would recognize colon26 increased as a result of administration of Compound (I).

The results demonstrate that Compound (I) induces the activation of immunocyte in vivo and has immunomodulation activity. [Example 7] Induction of cytokine production by Compound (I) in human peripheral blood mononuclear cell

Human peripheral blood mononuclear cells were introduced into a human complete medium (i.e., RPMI-1640 supplemented with 10% heat-inactivated FBS, 100 U/ml penicillin, and 100 μg/ml streptomycin) to prepare a cell suspension (1×10⁵ cells/ml). Compound (I), the EGFR tyrosine kinase inhibitor (i.e., AZD9291 or Erlotinib), or a cytokine-inducing positive control (i.e., Imiquimod) was added to the cell suspension. A control group to which the EGFR tyrosine kinase inhibitor and others were not added was also prepared (i.e., the non-treatment group). The culture solution was cultured in an incubator at 37° C. in the presence of 5% CO₂ for 2 days. The culture supernatant was recovered, and various types of cytokines were inspected with the use of the Bio-Plex Pro human cytokine assay kit.

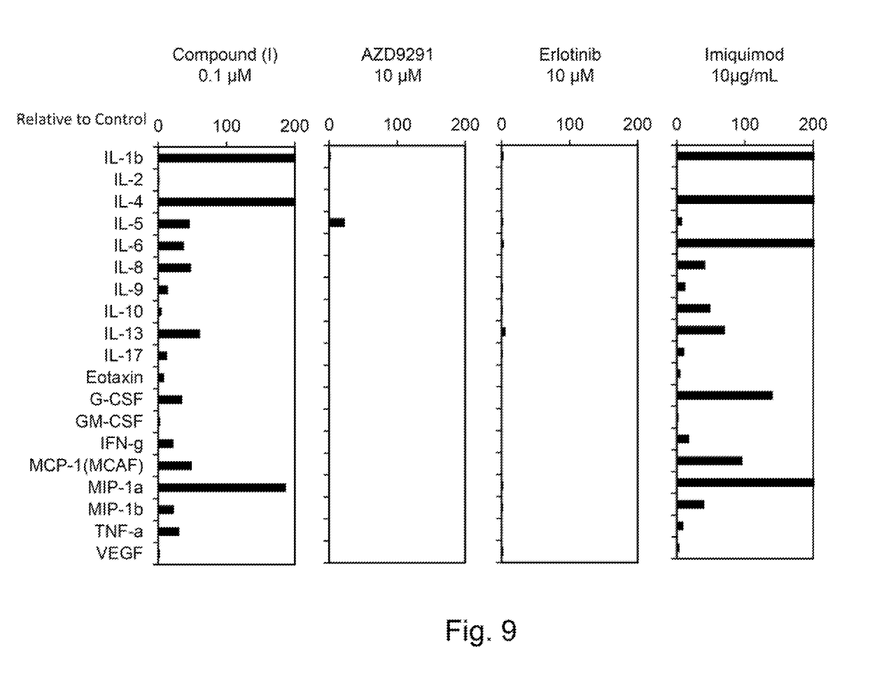

FIG. 9 shows a chart demonstrating the cytokine concentrations of the groups to which the compounds had been added, relative to the non-treatment group. As shown in the chart, Compound (I) has induced the production of various types of cytokines while such induction was not observed in the EGFR tyrosine kinase inhibitor equivalent thereto (i.e., AZD9291 or Erlotinib). The results demonstrate that Compound (I) has activity for inducing cytokine production and immunomodulation activity, which are inherent to Compound (I), and other EGFR tyrosine kinase inhibitors do not have such activities.

### [Reference Example 1] Influence of Compound (I) on the Growth of Mouse Melanoma Cell Line and Mouse Colon Cancer Cell Line in In Vitro Model

The mouse melanoma cell lines B16F10 and K1735M2 and the mouse colon cancer cell line MC38 were seeded in a 96-well plate at 3×10³ cells/well. Compound (I) or the EGFR tyrosine kinase inhibitor (i.e., Erlotinib, Afatinib, AZD9291, or Co1686 (Rociletinib)) that had been diluted to the given concentration was added 24 hours later. Thereafter, culture was conducted for 3 days, and the number of cells was determined using CellTiter-Glo2.0 (Promega, G9243). Compound (I) did not suppress the growth of any cells. Also, the other EGFR tyrosine kinase inhibitors did not suppress the growth of any cells.

### [Example 8] Influence of Compound (I) on the Growth of Mouse Melanoma Cell Line in In Vivo Model

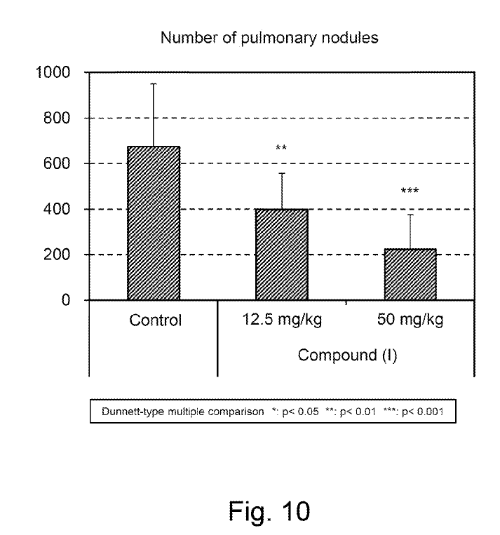

A cell suspension of the mouse melanoma cell line B16F10 was prepared with the use of PBS(-), and the cell suspension was injected intravenously into the mouse caudal portion at 5×10⁵ cells/mouse. Compound (I) was administered orally to the mouse model at 12.5 mg/kg or 50 mg/kg on the previous day of B16F10 implantation. The number of pulmonary metastatic nodules was evaluated 14 days after implantation (i.e., 15 days after drug administration). FIG. 10 shows the results of evaluation. In the group to which Compound (I) had been administered, the number of pulmonary metastatic nodules decreased in a dose-dependent manner, compared with the group to which Compound (I) had not been administered (i.e., the control group). On the basis of the results above and the results of Reference Example 1 such that the tumor growth inhibitory effects were not observed in the in vitro model, it was deduced that the decreased number of pulmonary metastatic nodules was achieved by the immunomodulation activity of Compound (I).

### [Example 9] Influence of Compound (I) on the Growth of Mouse Colon Cancer Cell Line in In Vivo Model

A cell suspension of the mouse colon cancer cell line MC38 was prepared with the use of PBS(-) and 50% Matrigel, and the cell suspension was injected subcutaneously into mice at 1×10⁶ cells/mouse. The mice were divided into groups when the average subcutaneous tumor volume reached approximately 50 mm³, Compound (I) was administered at 50 mg/kg or the anti-PD-1 antibody was administered at 100 μg/mouse, and the tumor volume was measured with the elapse of time. The tumor volume was determined on the basis of the longer diameter and the shorter diameter of the tumor measured percutaneously in accordance with Equation A shown below.

Tumor volume (mm³)=longer diameter (mm)×shorter diameter (mm)²/2 (Equation A)

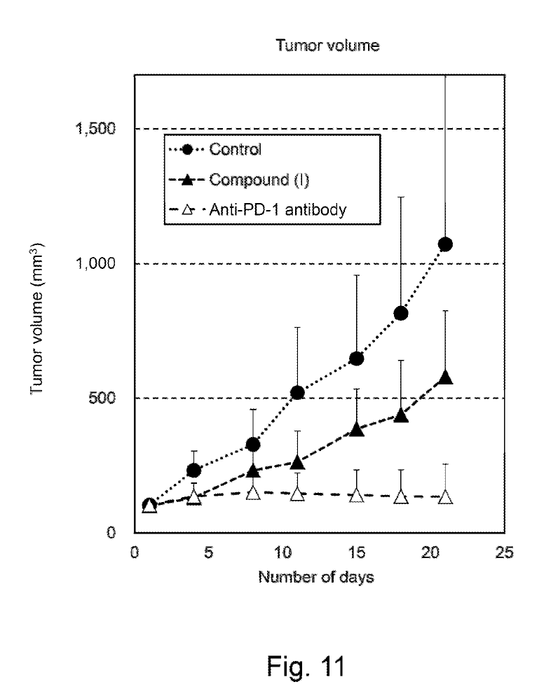

FIG. 11 shows changes in tumor volumes of the groups with the elapse of time. In the group to which Compound (I) had been administered, an extent of tumor growth inhibition was greater than that in the group to which Compound (I) had not been administered (i.e., the control group). On the basis of the results above and the results of Reference Example 1 such that the tumor growth inhibitory effects were not observed in the in vitro model, it was deduced that the tumor growth inhibition was achieved by the immunomodulation activity of Compound (I).

### [Example 10] Activity of Compound (I) for Potentiation of Antitumor Immune Response Inducer (Anti-PD-1 Antibody or Anti-PD-L1 Antibody) in In Vivo Model

A cell suspension of the mouse melanoma cell line K1735M2 was prepared with the use of PBS(-) and 50% Matrigel, and the cell suspension was injected subcutaneously into mice at 1×10⁶ cells/mouse. The mice were divided into groups based on their body weights 1 day after implantation, Compound (I) (50 mg/kg) or the anti-PD-1 antibody or anti-PD-L1 antibody (100 μg/mouse) was administered thereto alone or either the anti-PD-1 antibody or the anti-PD-L1 antibody was administered in combination with Compound (I), and the tumor volume was measured with the elapse of time. The tumor volume was determined on the basis of the longer diameter and the shorter diameter of the tumor measured percutaneously in accordance with Equation A shown above.

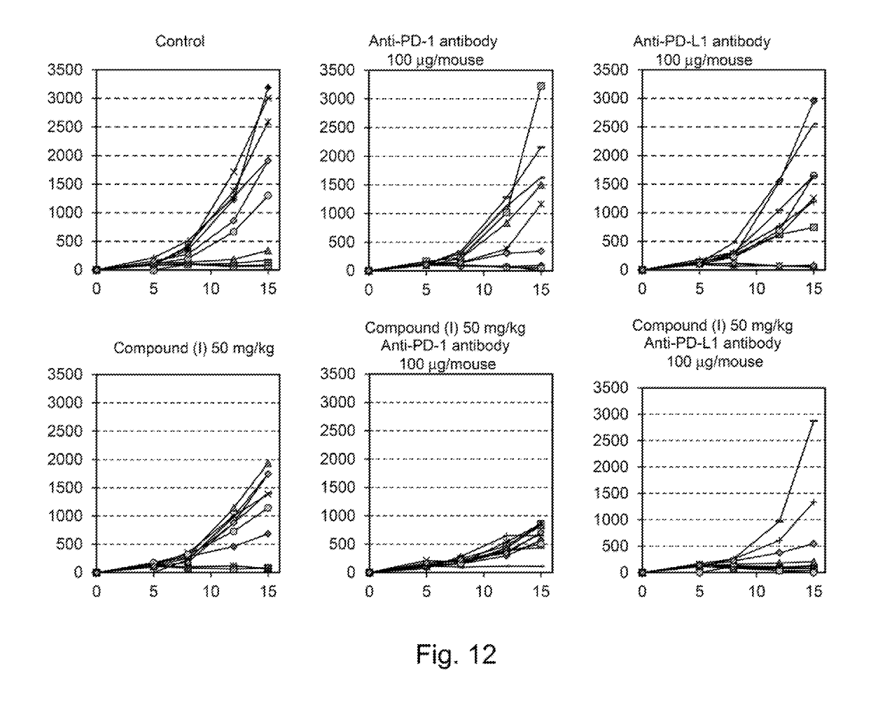

FIG. 12 shows changes in tumor volumes of individuals with the elapse of time. In the group to which Compound (I) had been administered alone and the group to which the anti-PD-1 antibody or the anti-PD-L1 antibody had been administered alone, an extent of tumor growth inhibition was not sufficient. In the group to which Compound (I) had been administered in combination with the anti-PD-1 antibody or the anti-PD-L1 antibody, however, an extent of tumor growth inhibition was significant. The results demonstrate that Compound (I) potentiates the immunomodulation activity of the anti-PD-1 antibody and the anti-PD-L1 antibody.

### [Example 11] Influence of Compound (I) on the Growth of Mouse Colon Cancer Cell Line and on Immunocyte in Tumor of In Vivo Model

A cell suspension of the mouse colon cancer cell line MC38 was prepared with the use of PBS(-) and 50% Matrigel, and the cell suspension was injected subcutaneously into mice at 1×10⁶ cells/mouse. The mice were divided into groups when the average subcutaneous tumor volume reached approximately 50 mm³, Compound (I) (50 mg/kg) or the anti-PD-1 antibody (50 μg/mouse) was administered thereto alone or Compound (I) (50 mg/kg) was administered in combination with the anti-PD-1 antibody (50 μg/mouse), and the tumor volume was measured with the elapse of time. The tumor volume was determined on the basis of the longer diameter and the shorter diameter of the tumor measured percutaneously in accordance with Equation A shown below.

Tumor volume (mm³)=longer diameter (mm)×shorter diameter (mm)²/2 (Equation A)

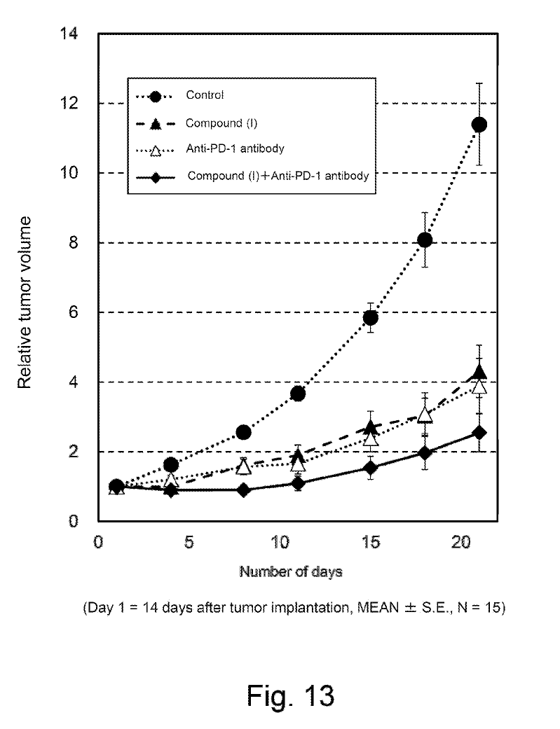

FIG. 13 shows changes in tumor volumes of the groups with the elapse of time. In the group to which Compound (I) had been administered, an extent of tumor growth inhibition was greater than that in the group to which Compound (I) had not been administered (i.e., the control group). On the basis of the results above and the results of Reference Example 1 such that the tumor growth inhibitory effects were not observed in the in vitro model, it was deduced that the tumor growth inhibition was achieved by the immunomodulation activity of Compound (I). It was also demonstrated that Compound (I) would potentiate the immunomodulation activity of the anti-PD-1 antibody.

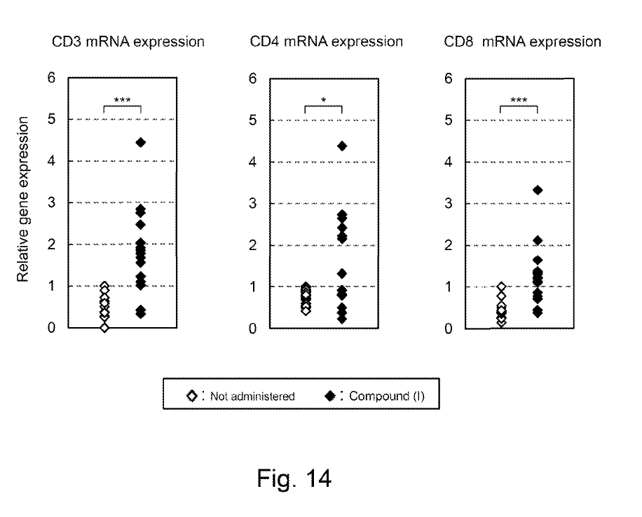

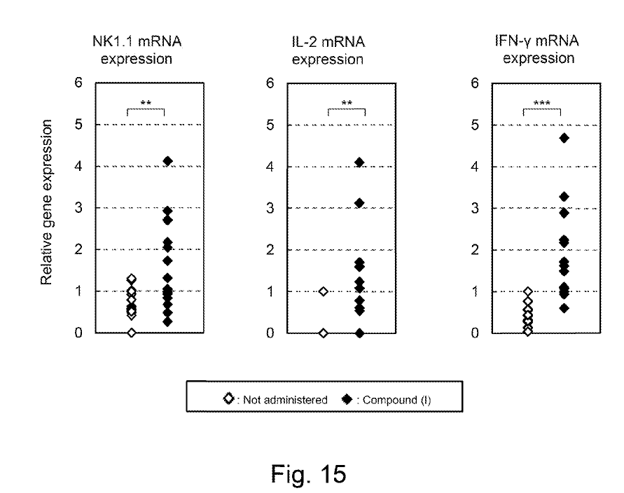

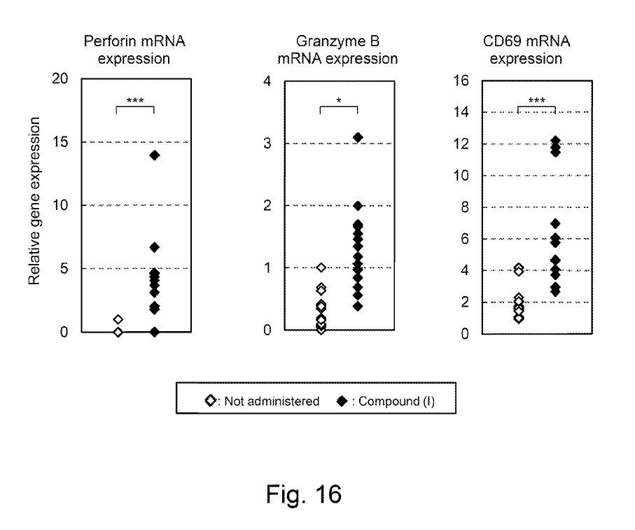

FIGS. 14 to 16 show changes in the immune-system-associated gene expression levels in tumors of the groups on the final day of administration of Compound (I). The tumor samples were obtained from the groups on the final day of administration of Compound (I) to prepare cDNA samples. The prepared cDNA samples were subjected to gene expression analysis via real-time PCR with the use of various immune-system-associated gene probes, and the results thereof were plotted. β-Actin was used as a control. Each plot indicates a gene expression level relative to a value of one of the controls. In the group to which Compound (I) had been administered, CD3, CD4, CD8, NK1.1, IL-2, IFN-γ, Perforin, Granzyme B, and CD69 gene expression levels increased, compared with those in the group to which Compound (I) had not been administered. On the basis of these results, it is deduced that Compound (I) exerts immunomodulation activity to increase the number of immunocytes to be exudated into tumors, leading to the tumor growth inhibition.

### INDUSTRIAL APPLICABILITY

The present invention enables the prevention or treatment of a disease that can be ameliorated via immunomodulation. The present invention also provides a novel method for the treatment of various infections, immunodeficiency, and tumors.

All publications, patents, and patent applications cited herein are incorporated herein by reference in their entirety.

1. An immunomodulator comprising (S)—N-(4-amino-5-(quinolin-3-yl)-6,7,8,9-tetrahydropyrimido[5,4-b]indolizin-8-yl)acrylamide or a salt thereof.

2. The immunomodulator according to claim 1, which activates T cells.

3. The immunomodulator according to claim 1, which induces IL-2 production.

4. The immunomodulator according to claim 1, which induces IFN production.

5. The immunomodulator according to claim 1, which induces immunocyte migration.

6. The immunomodulator according to claim 1, which induces exudation and accumulation of immunocytes in an affected area.

7. A method for modulating the immune system of a subject, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

8. A pharmaceutical composition for the prevention or treatment of an infection via immunomodulation, which comprises (S)—N-(4-amino-5-(quinolin-3-yl)-6,7,8,9-tetrahydropyrimido[5,4-b]indolizin-8-yl)acrylamide or a salt thereof.

9. The pharmaceutical composition according to claim 8, wherein the infection is an infection with a parasite.

10. The pharmaceutical composition according to claim 9, wherein the parasite is selected from the group consisting of trypanosomatid protozoa, malarial parasites, and Toxoplasma.

11. The pharmaceutical composition according to claim 8, wherein the infection is an infection with a bacterium.

12. The pharmaceutical composition according to claim 11, wherein the bacterium is selected from the group consisting of Streptococcus pneumoniae, Mycobacterium tuberculosis, Staphylococcus aureus, Bacillus anthracis, Vibrio cholerae, and Helicobacter pylori.

13. The pharmaceutical composition according to claim 8, wherein the infection is an infection with a virus.

14. The pharmaceutical composition according to claim 13, wherein the virus is selected from the group consisting of human T cell leukemia virus, papilloma virus, Epstein-Barr virus, cytomegalovirus, influenza virus, hepatitis B virus, and hepatitis C virus.

15. A method for the prevention or treatment of an infection of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

16. A pharmaceutical composition for the treatment of an immunodeficiency via immunomodulation, which comprises (S)—N-(4-amino-5-(quinolin-3-yl)-6,7,8,9-tetrahydropyrimido[5,4-b]indolizin-8-yl)acrylamide or a salt thereof.

17. The pharmaceutical composition according to claim 16, wherein the immunodeficiency is caused by an infection with HIV.

18. A method for the treatment of an immunodeficiency of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

19. A pharmaceutical composition for the prevention or treatment of a disease caused by an immune system weakened with age via immunomodulation, comprising (S)—N-(4-amino-5-(quinolin-3-yl)-6,7,8,9-tetrahydropyrimido[5,4-b]indolizin-8-yl)acrylamide or a salt thereof.

20. The pharmaceutical composition according to claim 19, wherein the disease caused by the weakened immune system is pneumonia.

21. A method for the prevention or treatment of a disease caused by an immune system weakened with age of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

22. A pharmaceutical composition for the prevention or treatment of a virus-associated tumor via immunomodulation, comprising (S)—N-(4-amino-5-(quinolin-3-yl)-6,7,8,9-tetrahydropyrimido[5,4-b]indolizin-8-yl)acrylamide or a salt thereof.

23. The pharmaceutical composition according to claim 22, wherein the virus-associated tumor is Burkitt's lymphoma, hepatic carcinoma, uterine cervix cancer, adult T cell leukemia, Kaposi's sarcoma, or head and neck cancer.

24. A method for the prevention or treatment of a virus-associated tumor of a subject via immunomodulation, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.

25. A pharmaceutical composition for the potentiation of the activity of a medicine used for preventing or treating a disease by acting on an immune system, comprising (S)—N-(4-amino-5-(quinolin-3-yl)-6,7,8,9-tetrahydropyrimido[5,4-b]indolizin-8-yl)acrylamide or a salt thereof.

26. The pharmaceutical composition according to claim 25, which is used for the potentiation of the activity of a vaccine to prevent an infection.

27. The pharmaceutical composition according to claim 25, which is used for the potentiation of the activity of an antiviral agent.

28. The pharmaceutical composition according to claim 25, which is used for the potentiation of the activity of an anti-PD-1 antibody or an anti-PD-L1 antibody.

29. The pharmaceutical composition according to claim 25, which is used for the potentiation of the activity of a cancer vaccine.

30. The pharmaceutical composition according to claim 25, which is used for the potentiation of the activity of an agent for inducing an antitumor immune response.

31. The pharmaceutical composition according to claim 30, wherein the agent for inducing an antitumor immune response is an anti-PD-1 antibody or an anti-PD-L1 antibody.

32. The pharmaceutical composition according to claim 31, wherein the agent for inducing an antitumor immune response is an anti-PD-1 antibody.

33. The pharmaceutical composition according to claim 31, wherein the agent for inducing an antitumor immune response is an anti-PD-L1 antibody.

34. A method for the potentiation of the activity of a medicine used for preventing or treating a disease of a subject by acting on an immune system, comprising administering a pharmaceutical composition comprising (S)—N-(4-amino-5-(quinolin-3-yl)-6,7,8,9-tetrahydropyrimido[5,4-b]indolizin-8-yl)acrylamide or a salt thereof to the subject.
